(19) United States
(12) Patent Application Publication
(10) Pub. No.: US 2016/0324760 A1
(43) Pub. Date: Nov. 10, 2016

(54) ALOPECIA TREATMENT COMPOSITION AND METHOD FOR PRODUCING THEREOF

(71) Applicant: ASSI TAL, NETANYA (IL)

(72) Inventor: ASSI TAL, NETANYA (IL)

(21) Appl. No.: 14/706,001

(22) Filed: May 7, 2015

(51) Int. Cl. A61K 8/97; A61K 8/98; A61K 8/67; A61K 8/92; A61Q 7/00

(52) U.S. Cl. CPC A61K 8/97; A61K 8/922; A61Q 7/00; A61K 8/678; A61K 8/988; A61K 2800/59

(57) ABSTRACT

An alopecia treatment composition, including a combination of ingredients of jasmine absolute, powder of Diospyros kaki sepals, Persea americana fruit oil, wheat germ oil, and lavender oil, to be applied onto a bald human area.

### TECHNICAL FIELD

The invention relates to the field of compositions for treatment. More particularly, the invention relates to a composition for the treatment of alopecia.

### BACKGROUND

Alopecia means loss of hair from the head or body. Alopecia can mean baldness, a term generally reserved for pattern alopecia or androgenic alopecia.

Baldness is the partial or complete lack of hair growth, and part of the wider topic of “hair thinning” The degree and pattern of baldness varies, but its most common cause is androgenic alopecia, alopecia androgenetica, or alopecia saborrheica, with the last term primarily used in Europe.

Pattern balding is distinct from alopecia areata, which commonly involves patchy hair loss. Extreme forms of alopecia areata are alopecia totalis, which involves the loss of all head hair, and the most extreme form, alopecia universalis, which involves the loss of all hair from the head and the body.

More than 95% of hair thinning in men is male pattern baldness, or androgenic alopecia. Male pattern baldness is characterized by hair receding from the lateral sides of the forehead (known as a “receding hairline”) and/or a thinning crown (balding to the area known as the “vertex”). Both become more pronounced until they eventually meet, leaving a horseshoe-shaped ring of hair around the back of the head.

The incidence of pattern baldness varies from population to population, and is based on genetic background. Environmental factors do not seem to affect this type of baldness greatly. One large scale study in Victoria, Australia, showed the prevalence of mid-frontal baldness increases with age and affects 73.5 percent of men and 57 percent of woman aged 80 and over. A rough rule of thumb is that the incidence of baldness in males corresponds to chronological age. For example, male pattern baldness (MPB) affects roughly 40 million men in the United States. Approximately 25 percent of men begin balding by age 30; two-thirds begin balding by age 60.

There is a 4 in 7 chance of receiving the baldness gene. Onset of hair loss sometimes begins as early as the end of puberty, and is mostly genetically determined. Both parents contribute to their offspring's likelihood of hair loss. Most likely, inheritance is technically “autosomal dominant with mixed penetrance”.

The trigger for this type of baldness is DHT, a powerful sex hormone, body- and facial-hair growth promoter that can affect the prostate as well as the hair located on the head. The mechanism by which DHT accomplishes this is not yet fully understood. Generally, DHT initiates a process of follicular miniaturization, in which the hair follicle begins to deteriorate. As a consequence, the hair's growth phase is shortened, and young, unpigmented vellus hair is prevented from growing and maturing into the deeply rooted and pigmented terminal hair that makes up 90 percent of the hair on our heads. In time, hair becomes thinner, and its overall volume is reduced so that it resembles fragile vellus hair until, finally, the follicle goes dormant and ceases producing hair completely.

Hair follicle growth occurs in cycles. Each cycle consists of a long growing phase (anagen), a short transitional phase (catagen) and a short resting phase (telogen). At the end of the resting phase, the hair falls out (exogen) and a new hair starts growing in the follicle beginning the cycle again.

Normally, about 40 (0-78 in men) hairs reach the end of their resting phase each day and fall out. When more than 100 hairs fall out per day, clinical hair loss (telogen effluvium) may occur. A disruption of the growing phase causes abnormal loss of anagen hairs (anagen effluvium).

Because they are not usually associated with an increased loss rate, male-pattern and female-pattern hair loss do not generally require testing.

The main cause of baldness, androgenic alopecia, also known as androgenetic alopecia, or male pattern baldness, is hair loss that occurs due to an underlying susceptibility of hair follicles to androgenic miniaturization. It is the most common cause of hair loss and will affect up to 70% of men and 40% of women at some point in their lifetime. Men typically present with hairline recession at the temples and vertex balding while women normally diffusely thin over the top of their scalps. Both genetic and environmental factors play a role, and many etiologies remain unknown.

Classic androgenic hair loss in males begins above the temples and vertex, or calvaria, of the scalp. As it progresses, a rim of hair at the sides and rear of the head remains. This has been referred to as a “Hippocratic wreath”, and rarely progresses to complete baldness. The Hamilton-Norwood scale has been developed to grade androgenic alopecia in males.

Female androgenic alopecia has been colloquially referred to as “female pattern baldness”, although its characteristics can occur in males as well. It more often causes diffuse thinning without hairline recession, and like its male counterpart, rarely leads to total hair loss. Female androgenic alopecia is usually characterized by diffuse crown thinning without a hairline recession. The Ludwig scale grades severity of androgenic alopecia in females.

The management of baldness is a multidisciplinary effort that spans the medical, pharmaceutical, food supplement, exercise, and cosmetics industries. Androgenic alopecia, alopecia areata, and telogen effluvium, are the primary nonscarring alopecias. The most common cause of hair loss in men is androgenic alopecia, the early stages of which can be slowed or reversed with medication, while more advanced cases may be amenable to hair transplantation. Alopecia areata presents as focal discoid patches of hair loss, and affects up to 2% of the U.S. population, occurring more often in children. Telogen effluvium can occur after stressful events, including severe illness, childbirth, or high fever, and can be seen with certain medications or deficiency of iron, particularly in females. Thyroid dysfunction, both when increased and decreased, can lead to specific patterns of hair loss.

Exercise can impact androgenic hair loss at least partially by affecting androgen and estrogen levels.

Quantification of androgen indices in response to exercise can be understood in certain categories: short versus long term, and anaerobic versus aerobic. These are indirect assays of exercise impact on hair loss, although the ability of exogenous androgen to worsen or precipitate miniaturization in the genetically predisposed is well documented. In cross-sectional analyses, aerobic exercisers have lower basal total and free testosterone compared to the sedentary. Anaerobic exercisers also have lower testosterone compared to the sedentary, but a slight increase in basal testosterone with resistance training over time.

WO2013149323, CA2772308 and KR1242736 disclose compositions for treatment. However, none of them disclose sepals of persimmon.

Thus, the methods described above have not yet provided satisfactory solutions to the problem of the treatment of alopecia.

### SUMMARY

One aspect of the invention is to provide a method and compositions for the treatment of alopecia.

Another aspect of the invention is to provide a method and compositions for the treatment of hair thinning or balding.

Another aspect of the invention is to provide a method and compositions for self-appliance to the skin for the treatment of alopecia.

Another aspect of the invention is to provide a solution to the above-mentioned and other problems of the prior art.

In one aspect, the invention is directed to an alopecia treatment composition, including a combination of ingredients of jasmine absolute, powder of Diospyros kaki sepals, Persea americana fruit oil, wheat germ oil, and lavender oil, to be applied onto a bald human area.

The reference numbers have been used to point out elements in the embodiments described and illustrated herein, in order to facilitate the understanding of the invention. They are meant to be merely illustrative, and not limiting. Also, the foregoing embodiments of the invention have been described and illustrated in conjunction with systems and methods thereof, which are meant to be merely illustrative, and not limiting.

### BRIEF DESCRIPTION OF THE DRAWINGS

Embodiments, features, and aspects of the invention are described herein in conjunction with the following drawings:

FIG. 1 is a schematic illustration of a bottle with a composition for a preliminary treatment of the skin of the head, and of a bottle with an alopecia treatment composition, according to one embodiment of the invention.

FIG. 2 is a schematic chart representing a preparation of a composition for a preliminary treatment of the skin of a head, to be used prior to an alopecia treatment composition according to one embodiment of the invention.

FIG. 3 is a schematic chart representing the preparation of a composition for the treatment of the skin of the head, in an alopecia treatment composition, according to one embodiment of the invention.

FIG. 4 is a schematic illustration of applying the composition for the preliminary treatment of the skin on the head of a user, according to one embodiment of the invention.

FIG. 5 is a schematic illustration of applying an alopecia treatment composition on the skin of the head, according to one embodiment of the invention.

FIG. 6 is a schematic illustration of rubbing an alopecia treatment composition into the skin of the head, according to one embodiment of the invention.

It should be understood that the drawings are not necessarily drawn to scale.

### DETAILED DESCRIPTION

The invention will be understood from the following detailed description of embodiments, which are meant to be descriptive and not limiting. For the sake of brevity, some well-known features, methods, systems, procedures, components, circuits, and so on, are not described in detail.

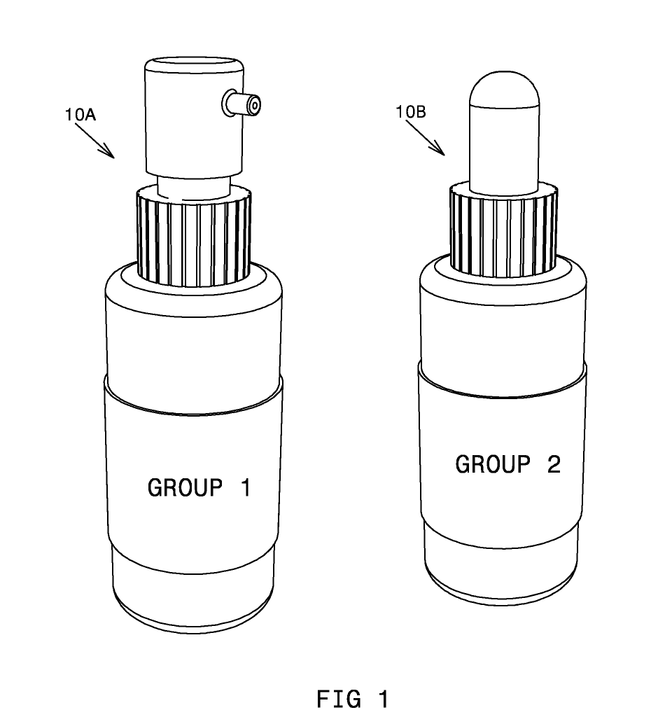

FIG. 1 is a schematic illustration of a bottle with a composition for a preliminary treatment of the skin of the head, and of a bottle with an alopecia treatment composition, according to one embodiment of the invention.

According to one embodiment of the invention, two bottles with two kinds of compositions are used for the treatment of alopecia. The first bottle contains a composition 10A, herein named “first composition” or “preliminary composition”, of “group 1”. According to this embodiment of the invention, the composition 10A of the substances of “group 1” is to be applied onto an area of skin treatment first, for the treating of alopecia. According to this embodiment, a bottle with another composition 10B is filled with “group 2” of substances that is to be applied later onto the area of the treated skin. The use of the composition 10A of “group 1” is for preparing the treated skin for the appliance of the second composition 10B of “group 2”. According to one embodiment of the invention, among other things, the composition 10A of “group 1” disinfects the treated skin, and expands the pores of the skin while cleaning it. By applying the composition 10A of “group 1”, the skin is washed from pollutants and is disinfected as a preliminary and preparatory action. The composition 10B of “group 2” in the other bottle contains active ingredients and substances for a treatment of alopecia.

According to one embodiment of the invention, the preliminary treatment of the skin of the head with the composition 10A of “group 1”, in addition to disinfecting the skin, also assists in encouraging blood flow to the area of treatment. An enhanced flow of the blood to the treated skin has many therapeutic advantages, ranging from supplying nutrient factors carried within the blood to the hair follicles, to supplying enhanced oxygen levels necessary for a healthy hair growth.

The combined treatment of washing the skin with the composition 10A of “group 1” and afterwards applying the composition 10B of “group 2” is shown to be effective for a treatment of alopecia and for stopping the process of balding. By using both compositions, firstly 10A of “group 1” and secondly 10B of “group 2”, a privately held clinical trial has proved that not only did the process of balding cease completely, but also new hair was growing in the group of patients suffering from baldness, covering the bald areas.

In a private test on different groups of people with different blood types, the following results have been achieved:

Each group had people with different types of baldness patterns. The trials showed successful results in all subjects, and a re-growth of hair in places where the hair was very thin before the beginning of the treatment with the compositions for treatment of alopecia according to one embodiment of the invention.

In another test of the compositions for treatment of alopecia according to one embodiment of the invention, the treatment of the patients was stopped before a full completion of recovery of the hair growth. Nevertheless, hair was continuing to grow, even though no further use had been made with the alopecia treatment composition. In this case, after the treatment of alopecia with the compositions according to one embodiment of the invention has been stopped, the hair of the patients continued to grow at places which were still bald at the time when the treatment stopped. This phenomenon is in contra distinction with the use of other materials and substances sold on the market and in the pharmacies. No other substance is claimed to continue the therapeutic process, even after the treatment has been stopped.

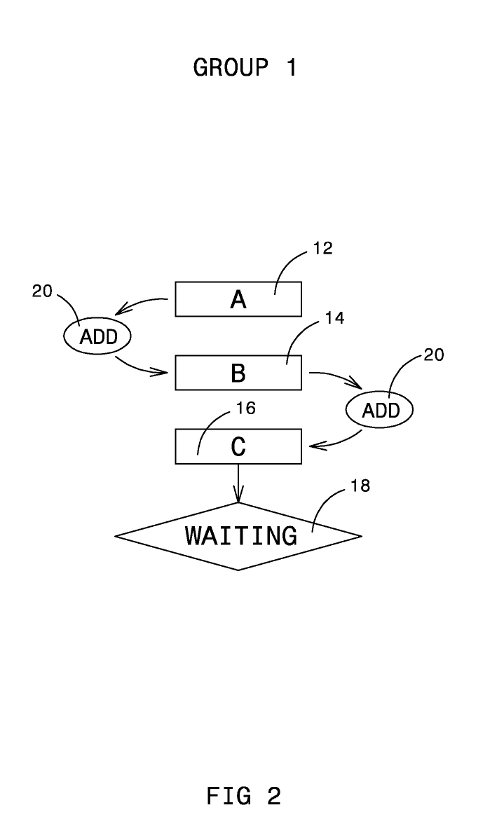

FIG. 2 is a schematic chart representing a preparation of a composition for a preliminary treatment of the skin of a head, to be used prior to an alopecia treatment composition according to one embodiment of the invention.

According to this embodiment, a preliminary treatment is to be applied onto the skin of the scalp. There are several purposes for applying the composition 10A of “group 1” first, among them: cleaning the skin, disinfecting it, opening the skin pores, and enhancing blood flow to the skin.

According to one embodiment of the invention, the composition 10A of “group 1” includes alcohol as a major ingredient. Ethanol is the preferred type of choice of the alcohol in the composition 10A, and is generally safe for the external use in washing an area of skin for the treatment of the scalp. A practical choice for using alcohol in the manufacturing of the composition 10A of “group 1” would be to use a liquid which is regularly sold at the chemical and pharmaceutical markets as 70% alcohol. (A choice between different concentrations of alcohol sold at the pharmaceutical market is possible here, ranging from 70% alcohol to a pure 100% alcohol). The other substances which are to be included in the composition of “group 1” according to one embodiment of the invention are tea tree oil 14, and propolis 16.

The schematic chart of FIG. 2 illustrates a method for producing the composition of “group 1”, according to one embodiment of the invention. According to this embodiment of the production method of the composition of “group 1” according to one embodiment of the invention, alcohol 12 is to be filled first into the production tank. The next step of production is adding 20 of tea tree oil 14 into the production tank which is filled with the alcohol. Then, the next step of production is adding 20 of propolis 16 to the composition in the production tank. After the three components (alcohol 12, tea tree oil 14, and propolis 16) have been inserted into the production tank, the next stage is to let the composition rest for about ten days. After waiting for a period of about ten days, the composition “group 1” is ready for use.

Tea tree oil 14 is produced from the leaves of a plant named Melaleuca alternifolia. The Melaleuca alternifolia tree is native to Southeast Queensland and the northeast coast of New South Wales, Australia. Tea tree oil is an essential oil with a fresh camphoraceous odor and a color that ranges from pale yellow to nearly colorless and clear. Tea tree oil has many medical uses, and it has suggested beneficial properties such as: antiviral, antibacterial, antifungal, and antiseptic. Its antiseptic qualities are previously known to be useful in treating skin blemishes such as acne, and it has other beneficial cosmetic properties. Although tea tree oil is normally extracted from Melaleuca alternifolia commercially, it can also be extracted from Melaleuca dissitiflora and Melaleuca linariifolia.

Propolis 16 is a resinous mixture that honey bees collect from tree buds, sap flows, or other botanical sources. It is used as a sealant for unwanted open spaces in the hive. Propolis is used for small gaps, while larger spaces are usually filled with beeswax. Its color varies depending on its botanical source, the most common being dark brown. Propolis is marketed by health food stores as a traditional medicine, and for its claimed beneficial effects on human health. Natural medicine practitioners use propolis for the relief of various conditions, including inflammations, viral diseases, ulcers, superficial burns or scalds. It is also believed to promote heart health and to strengthen the immune system. Depending upon its composition, propolis may show antibiotic and antifungal properties. Studies also indicate that it may be effective in treating skin burns.

In the preparation of the composition of “group 1” according to one embodiment of the invention, the relations between the three ingredients of the composition may be as follows: for a preparation of a bottle of 100 mL of “group 1”, six drops of tea tree oil 14 are inserted (a volume of about 0.4 mL of tea tree oil) with six grams of propolis into a volume of about 99 mL of ethanol.

According to one mode of preparation for a bottle of 100 mL of the composition of “group 1” according to an embodiment of the invention, a volume of about 99 mL of alcohol is poured into a bottle, then a volume of about 0.3 mL-0.4 mL of tea tree oil is dripped into the alcohol, and then a piece of about 6 grams of propolis is inserted. After waiting for about 10 days, the composition of “group 1” is ready for use.

According to another embodiment of the invention, for a preparation of a bottle of 100 mL of the composition 10A composed of the substances from “group 1”, a volume of between 95 mL to 99.5 mL of alcohol is used, with a volume of between 0.2 mL to 4.9 mL of tea tree oil, and about 0.2 grams to 12 grams of propolis.

The combination of alcohol with tea tree oil 14 and propolis 16 in the composition of “group 1” produce a very effective antiseptic, disinfecting, blood flow stimulating and skin pores expanding composition. The effect of the composition 10A of “group 1” on the treated skin according to the invention, is very efficient for cleaning a greasy scalp skin filled with sebum (an oily, waxy substance, that is secreted from the sebaceous glands), for a successful treatment of alopecia. The condition of alopecia saborrheica is characterized in an excess of sebum that is accumulated in the skin of the scalp, and is a main factor in the majority of alopecia cases. The secreted sebum, a very oily and waxy substance made of triglyceride oils, wax and squalene, can be easily washed off with the produced composition 10A of the “group 1” of substances according to one embodiment of the invention. The disinfecting properties of the composition 10A of “group 1” is useful for removing the sebum from the skin and cleaning the skin pores, which is helpful in allowing more oxygen to reach the follicles inside the skin and letting the skin breathe a lot better. This is a beneficial property of the composition 10A from the substances of “group 1”, which contributes to a successful treatment of a vast variety of balding problems, according to an embodiment of the invention.

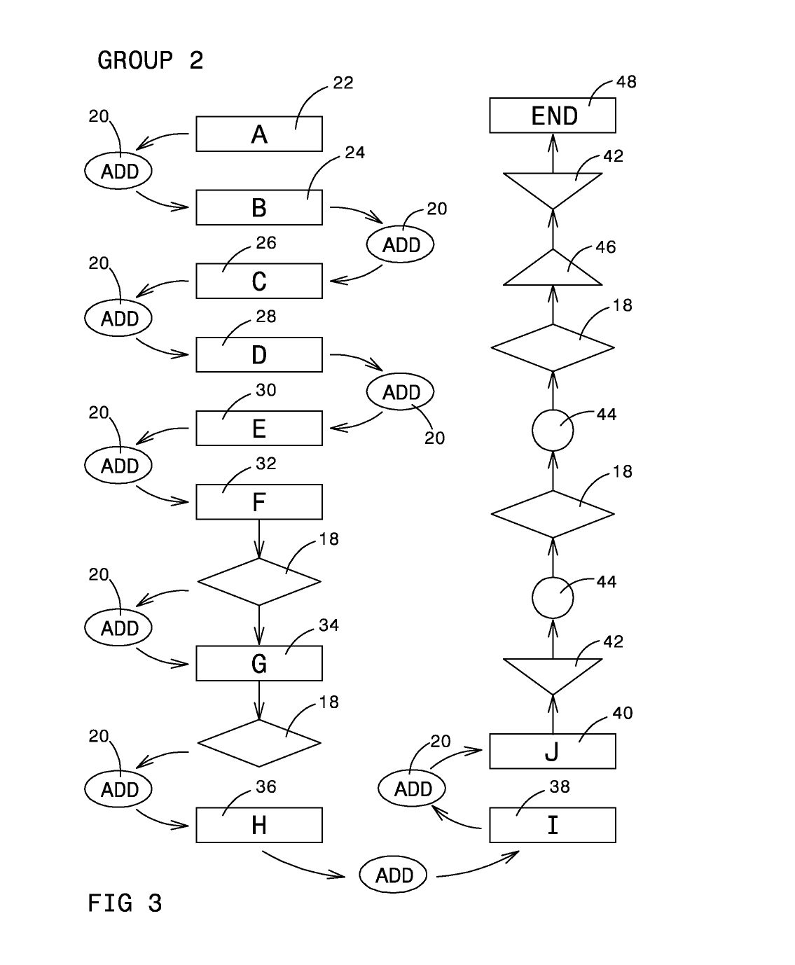

FIG. 3 is a schematic chart representing the preparation of a composition for the treatment of the skin of the head, in an alopecia treatment composition according to one embodiment of the invention.

According to one embodiment of the invention, the composition 10B of “group 2” for a treatment of alopecia may be made by adding the following substances according to the following procedure:

Into a production vessel, a powder of Diospyros kaki sepals 22 (also known as the fruit of persimmon of the genus Diospyros) is to be inserted. The next step is adding 20 of wheat germ oil 24 to the vessel. Then adding 20 lavender oil 26. Then adding 20 Leptospermum scoparium oil 28 (also known as manuka oil) into the vessel. And then, adding 20 Olea europaea fruit oil 30 (known as olive oil) into the vessel. Then, adding 20 vitamin E 32. Then, waiting (18) for about two to three days, letting the composition rest in the vessel. Then adding 20 eucalyptus oil and Cananga odorata oil 34, and waiting (18) for another two days, allowing chemical bonds to form in the composition. Then, adding 20 Persea americana fruit oil 36 (known as avocado oil), jasmine absolute 38, and almond oil 40 into the vessel. Then, the vessel is hermetically sealed (42). Next, the composition in the vessel is stirred in repetitions of about five minutes for each stirring, every five hours, for a period of about one week 44. Then waiting 18 for about five hours, and again stirring in repetitions of about five minutes per stir session, every twelve hours, for one week 44. Then, waiting for about twelve hours, and then performing an inspection of the composition 46. After the inspection, the next step (42) is sealing in a bottle. And then, the composition for treatment of alopecia 48 according to one embodiment of the invention is ready for use.

According to an embodiment of the invention, the composition “group 2” for a treatment of alopecia is to be prepared according to the following procedure: For preparation of about 100 mL of the composition “group 2”, in room temperature of about 22 degrees Celsius, a powder of about 4 g of persimmon sepals 22 is inserted into an empty bottle. Then, about 23 mL of wheat germ oil 24 is inserted into the bottle. Then about 0.6 mL of lavender oil 26 is added. Then about 0.25 mL of manuka oil 28 is inserted into the bottle. Then, about 5 mL olive oil 30 is added to the bottle. Then, about 0.75 mL of vitamin E is added. Then, the bottle is left untouched, while waiting for about two to three days for letting the substances in the bottle react. Then, adding 20 about 0.1 mL of eucalyptus oil to the bottle, and about 0.25 mL of ylang-ylang oil 34, and then waiting 18 for about two days, allowing the bottle to rest untouched. Then, adding 20 about 7 mL avocado oil 36, about 4.8 mL jasmine absolute 38, and then, filling the bottle until it is full with almond oil 40. Next (42), the bottle is sealed. And then, the composition in the bottle is stirred every 5 hours for 5 minutes each time, for about one week. And then the composition is stirred every 12 hours for 5 minutes each time, for another week. Then, waiting 18 for another week while letting the bottle stay untouched. At this point, the bottle may be opened only for performing an inspection to look at the color of the composition and for observing in case of an irregular composition. After a successful inspection, the bottle is sealed, and a 100 mL of “group 2” is ready for the treatment of alopecia according to an embodiment of the invention.

According to another embodiment of the invention, adding the substances may be performed in a different order than that specified above. Moreover, the quantities of the substances may vary somewhat from the quantities of the substances which were named above.

According to another embodiment of the invention, the composition “group 2” for a treatment of alopecia is to be prepared according to the following: For preparation of 100 g of the composition “group 2”, a powder of 2 g-8 g of persimmon sepals 22 is inserted into a vessel with 5 g-40 g of wheat germ oil 24, with 0.2 g-3 g of lavender oil 26, with 0.1 g-3 g of manuka oil 28, with 2 g-10 g of olive oil 30, with 0.3 g-3 g of vitamin E. Then, after waiting, 0.05 g-2 g of eucalyptus oil is added to the vessel, with 0.1 g-2 g of ylang-ylang oil 34. Then after waiting, adding 3 g-20 g of avocado oil 36, with 2 g-10 g of jasmine absolute 38, and then filling the remainder of the vessel with almond oil 40.

According to one embodiment of the invention, some substances may be omitted from the composition of “group 2”. Accordingly, not all the specified substances are essentially included in “group 2”. Other substitutes may be used for the replacement of some of the substances specified above in “group 2”.

For a matter of clarification, it is said that the substances that may be used for the preparation of the composition “group 2” may be named in synonymous names, for example:
Diospyros kaki may refer to the fruit of persimmon;
Leptospermum scoparium oil may refer to manuka oil;
Olea europaea fruit oil may refer to olive oil;
Cananga odorata oil may refer to ylang-ylang essential oil;
Persea americana fruit oil may refer to avocado oil; and
Lavandula may refer to Lavender.

The term “sieve grinder” refers herein to a sieve, used for grinding.

The sepal is rubbed back and forth, or by a rotary motion, on a sieve. The rubbing against the strings of the sieve grinds the sepal, together with allowing physical fall thereof only after the size of the powder is smaller than the size of the holes of the sieve.

The thinner the solid particles of the powder are, the greater is the penetration of the composition into the skin, since the powder is not soluble by the liquid ingredients.

According to one embodiment of the invention, the Diospyros kaki sepals are to be grinded by a sieve. The preferred instrument for a clean and smooth grinding of the sepals is a silk sieve: a very delicate sieve made of silk, for obtaining a smooth and thin powder product. A preferred type of silk sieve for grinding the sepals of the persimmon is a very dense sieve (e.g., an 80% sieve). The resulting powder will be much thinner and delicate by grinding the sepals with a dense silk sieve, and hence the effectiveness of the treatment with the prepared composition according to one embodiment of the invention will increase.

The Diospyros kaki sepals are the four sepals that stem from the base of the persimmon fruit. According to one embodiment of the invention, the four sepals are initially picked from the persimmon fruit, and then they are ground with the silk sieve into a thin powder, before they are added to the composition 10B.

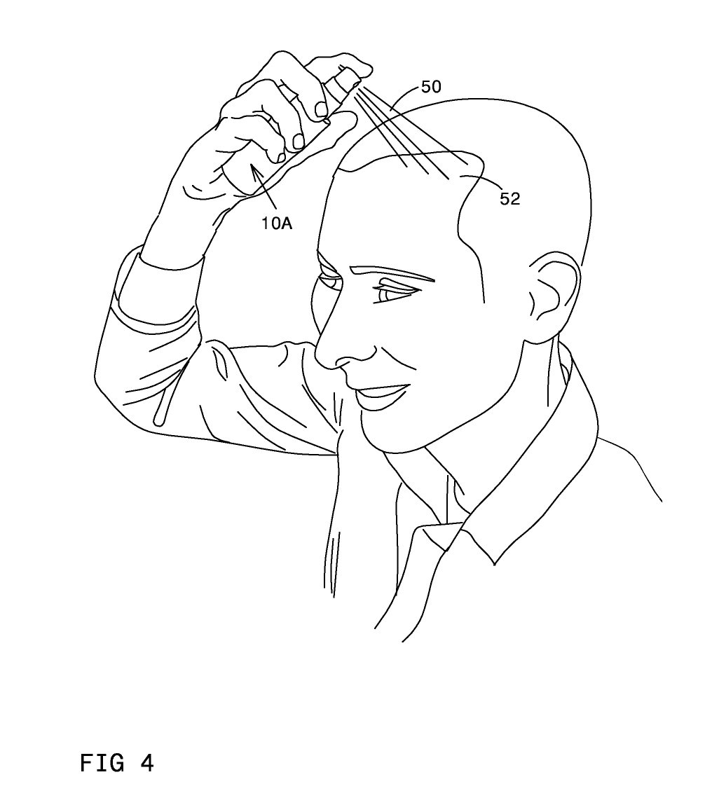

FIG. 4 is a schematic illustration of applying the composition for the preliminary treatment of the skin on the head of a user, according to one embodiment of the invention.

According to one embodiment of the invention, a bottle is filled with the composition 10A of “group 1” of substances for application onto the skin. The composition 10A of the substances of “group 1” is to be applied to a thinning hair area for cleaning the inner side of the skin. The washing effect of the composition 10A of “group 1” makes it suitable to apply it to an area of skin for the treatment of alopecia, as the composition 10A will wash pollutants and disinfect treated skin.

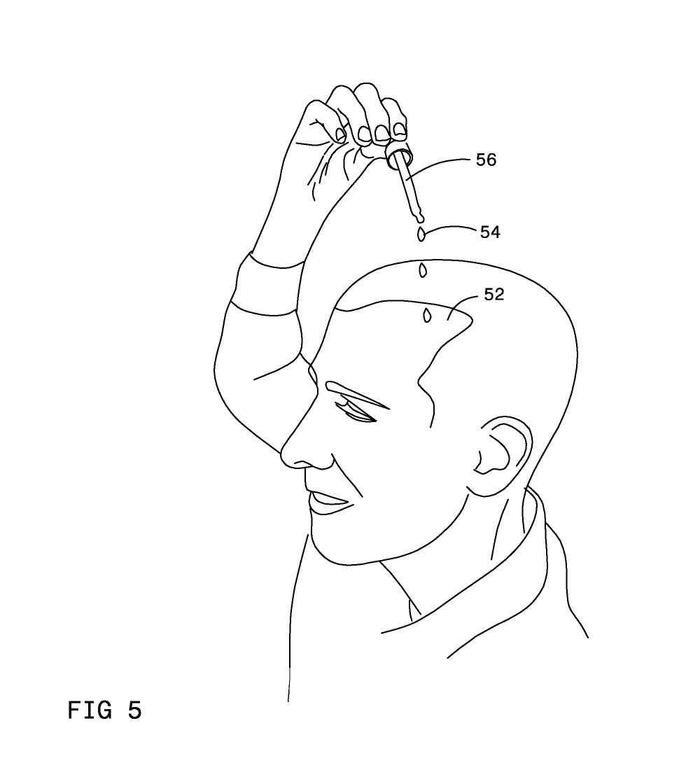

According to one embodiment of the invention, the composition of the substances of “group 1” is to be applied onto the skin of the scalp by spraying the composition on the head of a user. The first composition 10A is to be applied to the skin by the spraying of “group 1” of substances 50 onto an area of alopecia 52. The user may hold a bottle of “group 1” of composition 10A in his hand and spray enough of the liquid of the composition 10A to wet the treated area of alopecia 52. According to one embodiment of the invention, the substances of “group 1” react on the treated area of alopecia 52 to clean, disinfect and open the pores in the skin to prepare the treated area for the second treatment with the composition of “group 2” (as described in FIG. 5). The use of the composition of “group 1” should be done with caution, to avoid any contact of the substance with the eyes of a user. The reaction of “group 1” with the treated area of alopecia 52 cleans the treated skin from oils, from greasy substances in the skin, from sebum, and other waxy substances, and prepares the area for the next step of the treatment in accordance with the invention.

According to one embodiment of the invention, spraying of two doses from “group 1” permits the wetting of an entire area of treatment of alopecia, and after spraying an area of alopecia 52 the user will massage the treated skin with his hands for a better applicability.

FIG. 5 is a schematic illustration of applying an alopecia treatment composition on the skin of the head, according to one embodiment of the invention.

According to one embodiment of the invention, the composition 10B from the “group 2” of substances is to be applied onto an area of alopecia 52. By applying “group 2” of substances 54 with a dropper 56, the user implements the alopecia treatment composition 10B onto an area of alopecia 52.

According to an embodiment of the invention, the substances of “group 2” for the treatment of alopecia may be applied to an area of alopecia 52 after the treatment with a first composition 10A of the substances of “group 1”. According to this embodiment of the invention, the treatment with the first composition 10A of “group 1” prepares and cleans the treated area for the application of composition 10B of “group 2” to the treated area of alopecia 52.

According to another embodiment of the invention, the application of the composition 10B of “group 2” suffices for a treatment of alopecia, and can be applied on an area of alopecia 52 without prior treatment of the skin with any other composition. In this case, the applying of “group 2” of substances 54 preferably takes place after a thorough wash of the scalp with clean hot water.

According to one embodiment of the invention, a quantity of 1 mL-6 mL of the composition 10B of “group 2” is to be applied onto a treated area of alopecia 52.

In the case wherein the patient has relatively full hair, the used quantity of the composition 10B of “group 2” of substances should be relatively large for the composition to penetrate through the dense hair to reach the skin of the scalp. In a case wherein the patient has dense hair, a quantity of about 2.5 mL-6 mL is to be used for the composition 10B of “group 2” to be applied on the skin in a sufficient quantity.

In the case wherein the hair of a patient is very thin or even completely bold, a relatively small quantity of the composition 10B of “group 2” in enough to cover an area of alopecia 52. In this case, a quantity of about 1 mL-2.5 mL of the composition 10B of “group 2” may be enough for a treatment of an area of alopecia 52.

According to an embodiment of the invention, a quantity of about 2.5 mL of the composition 10B of “group 2” is to be applied on an area of alopecia 52 for the composition to cover the area for treatment.

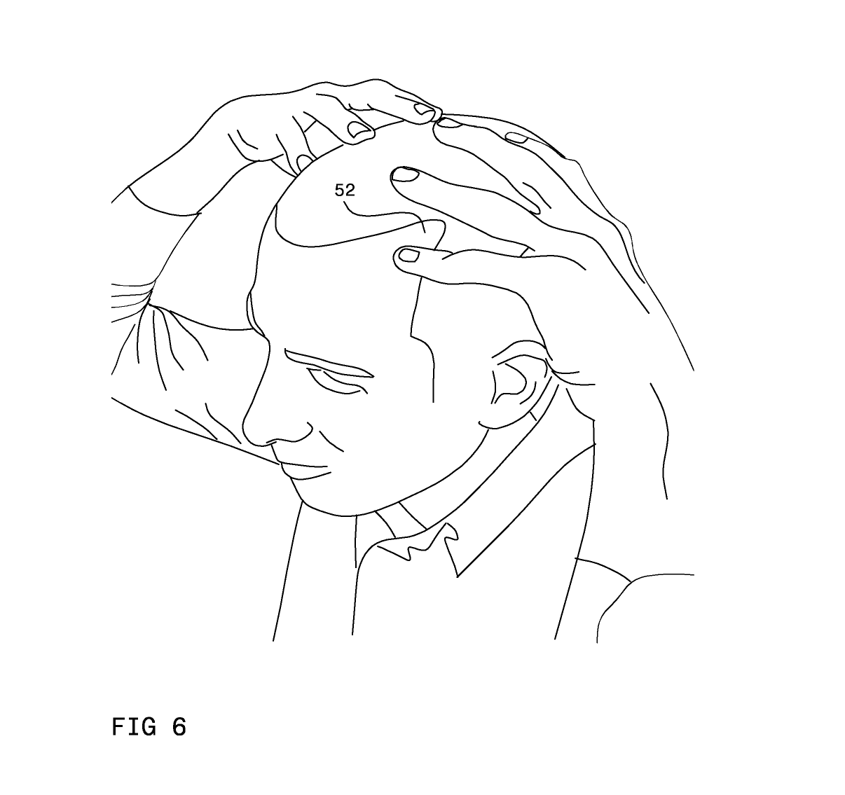

FIG. 6 is a schematic illustration of rubbing an alopecia treatment composition into the skin of the head, according to one embodiment of the invention.

According to this embodiment of the invention, after the second composition 10B for the treatment of the skin is applied to the skin of the head, the user is to rub the composition material gently on the skin of the head with his hands, by massaging the area affected by alopecia.

According to an embodiment of the invention, the composition 10B of “group 2” is to be rubbed and spread on the area of alopecia 52 by the hands. The composition 10B of “group 2” penetrates the open pores of the treated skin (after a preferred initial treatment with the composition 10A of “group 1”), and react on the follicles to treat the condition of alopecia according to an embodiment of the invention.

According to an embodiment of the invention, the treatment of alopecia with the compositions 10A and 10B is to be done daily.

According to an embodiment of the invention, the treatment of alopecia with the compositions 10A and 10B is to be done in the evening before going to sleep. It is preferred that after the application of the composition 10B of “group 2” on an area of alopecia 52, a user will massage the area for about 3-5 minutes with his hands, to help the composition to be absorbed in the skin. After the user has massaged the area of alopecia 52 with his hands to spread the composition 10B of “group 2” on the skin, it is preferred that a hat or a plastic bag be put on to cover the area. A hat or a plastic bag that covers the area of alopecia 52 with the composition 10B spread on the skin, allows keeping the composition on the surface of the skin during the night.

According to an embodiment of the invention, the treatment of alopecia with the compositions 10A and 10B may take from 4 months to 12 months, in a daily treatment, until a successful result of hair growth is achieved. According to this embodiment, it is preferred that the treatment with the composition 10B take place every evening before going to sleep, in order to allow the composition 10B to stay on the treated skin during the night.

In summary, in one aspect, the invention is directed to an alopecia treatment composition (10B), comprising:

a combination of ingredients of:
jasmine absolute (38);
powder of Diospyros kaki sepals (22);
Persea americana fruit oil (36);
wheat germ oil (24); and
lavender oil (26);
   to be applied onto a bald human area.

For 100 grams of the composition, the ingredients may comprise the following amounts:
the jasmine absolute: 2-10 grams;
the powder of Diospyros kaki sepals: 2-8 grams;
the Persea americana fruit oil: 3-20 grams;
the wheat germ oil: 5-40 grams; and
the lavender oil: 0.2-3 grams.

The alopecia treatment composition (10B) may further comprise vitamin E (32).

The alopecia treatment composition (10B) preferably is for being applied onto the area of baldness after treatment of a preliminary composition (10A) comprising alcohol (12).

The preliminary composition (10A) may further comprise tea tree oil (14).

The preliminary composition (10A) may further comprise propolis (16).

The alopecia treatment composition (10B) may further comprise almond oil (40).

For 100 grams of the composition (10B), the amount of vitamin E (32) may comprise 0.3-3 grams.

The composition (10B) may be prepared at room temperature, and

wherein for 100 milliliters of the composition (10B), the ingredients may comprise the following amounts:
the jasmine absolute: 4.8 milliliters;
the powder of Diospyros kaki sepals: 4 grams;
the Persea americana fruit oil: 7 milliliters;
the wheat germ oil: 23 milliliters; and
the lavender oil: 0.6 milliliters;
   and wherein for the 100 milliliters of the composition (10B), the composition (10B) may further comprise the following ingredients in the following amounts:
vitamin E: 0.75 milliliters;
almond oil: 56.25 milliliters;
manuka oil: 0.25 milliliters;
olive oil: 5 milliliters;
eucalyptus oil: 0.1 milliliters; and
ylang-ylang oil: 0.25 milliliters;
   and wherein the composition (10B) is mixed every 5 hours for 5 minutes for at least one week.

The alopecia treatment composition (10B) preferably is for being applied onto a bald human area once per day.

The alopecia treatment composition (10B) preferably is for being applied daily onto a bald human area for at least four months.

In another aspect, the invention is directed to an alopecia treatment composition (10B), comprising:

a combination of ingredients of:
jasmine absolute (38); and
powder of Diospyros kaki sepals (22);
   to be applied onto a bald area of human skin,
   wherein solid particles of the powder of Diospyros kaki sepals (22) are sufficiently thin for penetrating into the skin.

The alopecia treatment composition (10B) may further comprise:
Persea americana fruit oil (36);
wheat germ oil (24);
lavender oil (26);
vitamin E (32); and
almond oil (40).

The alopecia treatment composition (10B) may further comprise:
manuka oil;
olive oil;
eucalyptus oil; and
ylang-ylang oil.

In another aspect, the invention is directed to a method for producing an alopecia treatment composition (10B), the method comprising the steps of adding the following ingredients:
jasmine absolute (38);
powder of Diospyros kaki sepals (22);
Persea americana fruit oil (36);
wheat germ oil (24); and
lavender oil (26);
   thereby producing a composition to be applied onto a bald human area.

The method may further comprise the steps of adding:
vitamin E (32); and
almond oil (40).

The method may further comprise the steps of mixing the ingredients at room temperature every 5 hours for 5 minutes for at least one week.

The method may further comprise the step of producing a preliminary composition (10A) by the step of adding alcohol (12) thereto.

The method may further comprise the step of adding tea tree oil (14) to the first composition (10A).

The method may further comprise the step of adding propolis (16) to the first composition (10A).

The method may further comprise the step of grinding the Diospyros kaki sepals by a sieve grinder.

The sieve grinder may comprise a silk sieve.

For 100 grams of the composition, the ingredients comprise the following amounts:
the jasmine absolute: 2-10 grams;
the powder of Diospyros kaki sepals: 2-8 grams;
the Persea americana fruit oil: 3-20 grams;
the wheat germ oil 5-40 grams; and
the lavender oil 0.2-3 grams.

In the figures and/or description herein, the following reference numerals (Reference Signs List) have been mentioned:
numeral 10A denotes a composition of “group 1” of substances;
numeral 10B denotes a composition of “group 2” of substances;
numeral 12 denotes alcohol;
numeral 14 denotes tea tree oil;
numeral 16 denotes propolis;
numeral 18 denotes a waiting period;
numeral 20 denotes adding a substance;
numeral 22 denotes powder of Diospyros kaki sepals;
numeral 24 denotes wheat germ oil;
numeral 26 denotes lavender oil;
numeral 28 denotes Leptospermum scoparium oil (manuka oil);
numeral 30 denotes Olea europaea fruit oil (olive oil);
numeral 32 denotes vitamin E;
numeral 34 denotes eucalyptus oil and Cananga odorata oil;
numeral 36 denotes Persea americana fruit oil;
numeral 38 denotes jasmine absolute;
numeral 40 denotes almond oil;
numeral 42 denotes the step of sealing in a bottle;
numeral 44 denotes repetitions of stirrings for five minutes for one week;
numeral 46 denotes inspection of the composition;
numeral 48 denotes composition for treatment of alopecia;
numeral 50 denotes spraying of “group 1” of substances;
numeral 52 denotes an area of alopecia;
numeral 54 denotes “group 2” of substances; and
numeral 56 denotes a dropper.

The foregoing description and illustrations of the embodiments of the invention has been presented for the purposes of illustration. It is not intended to be exhaustive or to limit the invention to the above description in any form.

Any term that has been defined above and used in the claims, should to be interpreted according to this definition.

The reference numbers in the claims are not a part of the claims, but rather used for facilitating the reading thereof. These reference numbers should not be interpreted as limiting the claims in any form.

What is claimed is:

1. An alopecia treatment composition, comprising:
a combination of ingredients of:
jasmine absolute;
powder of Diospyros kaki sepals;
Persea americana fruit oil;
wheat germ oil; and
lavender oil;
to be applied onto a bald human area.

2. An alopecia treatment composition according to claim 1, characterized in that for 100 grams of said composition, said ingredients comprise the following amounts:
said jasmine absolute: 2-10 grams;
said powder of Diospyros kaki sepals: 2-8 grams;
said Persea americana fruit oil: 3-20 grams;
said wheat germ oil: 5-40 grams; and
said lavender oil: 0.2-3 grams.

3. An alopecia treatment composition according to claim 1, further comprising vitamin E (32).

4. An alopecia treatment composition according to claim 1, for being applied onto the bald human area after treatment of a preliminary composition comprising alcohol.

5. An alopecia treatment composition according to claim 4, wherein said preliminary composition further comprises tea tree oil.

6. An alopecia treatment composition according to claim 4, wherein said preliminary composition further comprises propolis.

7. An alopecia treatment composition according to claim 1, further comprising almond oil.

8. An alopecia treatment composition according to claim 3, wherein for 100 grams of said composition, the amount of said vitamin E (32) comprises 0.3-3 grams.

9. An alopecia treatment composition according to claim 1, wherein said composition is prepared at room temperature, and
wherein for 100 milliliters of said composition, said ingredients comprise the following amounts:
said jasmine absolute: 4.8 milliliters;
said powder of Diospyros kaki sepals: 4 grams;
said Persea americana fruit oil: 7 milliliters;
said wheat germ oil: 23 milliliters; and
said lavender oil: 0.6 milliliters;
and wherein for the 100 milliliters of said composition, said composition further comprises the following ingredients in the following amounts:
vitamin E: 0.75 milliliters;
almond oil: 56.25 milliliters;
manuka oil: 0.25 milliliters;
olive oil: 5 milliliters;
eucalyptus oil: 0.1 milliliters; and
ylang-ylang oil: 0.25 milliliters;
and wherein said composition is mixed every 5 hours for 5 minutes for at least one week.

10. An alopecia treatment composition, comprising:
a combination of ingredients of:
jasmine absolute; and
powder of Diospyros kaki sepals;
to be applied onto a bald area of human skin,
wherein solid particles of said powder of Diospyros kaki sepals are sufficiently thin for penetrating into the skin.

11. An alopecia treatment composition according to claim 10, further comprising:
Persea americana fruit oil;
wheat germ oil;
lavender oil;
vitamin E (32); and
almond oil.

12. An alopecia treatment composition according to claim 11, further comprising:
manuka oil;
olive oil;
eucalyptus oil; and
ylang-ylang oil.

13. A method for producing an alopecia treatment composition, the method comprising the steps of adding the following ingredients:
jasmine absolute;
powder of Diospyros kaki sepals;
Persea americana fruit oil;
wheat germ oil; and
lavender oil;
thereby producing a composition to be applied onto a bald human area.

14. A method according to claim 13, further comprising the steps of adding:
vitamin E (32); and
almond oil.

15. A method according to claim 13, further comprising the steps of mixing said ingredients at room temperature every 5 hours for 5 minutes for at least one week.

16. A method according to claim 13, further comprising the step of producing a preliminary composition by the step of adding alcohol thereto.

17. A method according to claim 16, further comprising the step of adding tea tree oil to said first composition.

18. A method according to claim 16, further comprising the step of adding propolis to said first composition.

19. A method according to claim 13, further comprising the step of grinding said Diospyros kaki sepals by a sieve grinder.

20. A method according to claim 19, wherein said sieve grinder comprises a silk sieve.
